(19) United States
(12) Patent Application Publication
(10) Pub. No.: US 2015/0132227 A1
(43) Pub. Date: May 14, 2015

(54) NOVEL ECHOGENIC CONTRAST AGENTS

(71) Applicant: UNIVERSITY OF UTAH RESEARCH FOUNDATION, Salt Lake City, UT (US)

(72) Inventors: Leonard F. Pease, Bountiful, UT (US); Hedieh Saffari, Salt Lake City, UT (US); Kathryn Peterson, Salt Lake City, UT (US); Kristin Leiferman, Salt Lake City, UT (US); Anne Kennedy, Salt Lake City, UT (US); Russell Morris Condie, Salt Lake City, UT (US); Gerald Gleich, Salt Lake City, UT (US)

(21) Appl. No.: 14/395,407

(22) PCT Filed: Apr. 18, 2013
(86) PCT No.: PCT/US2013/037194

§ 371 Date: Oct. 17, 2014

Related U.S. Application Data
(60) Provisional application No. 61/687,106, filed on Apr. 18, 2012.
(60) Provisional application No. 61/687,801, filed on May 2, 2012.
(60) Provisional application No. 61/796,059, filed on Nov. 1, 2012.
(60) Provisional application No. 61/790,925, filed on Mar. 15, 2013.

(51) Int. Cl. A61K 49/22

(52) U.S. Cl. CPC A61K 49/221; A61K 49/22
USPC 424/9.5; 530/391.3

(57) ABSTRACT

Disclosed are compositions and methods for related to echogenic contrast agents. Also disclosed are methods and compositions for diagnosing eosinophilic esophagitis in a subject. Also disclosed are methods for producing a medical image of a tissue. Also disclosed are methods of monitoring the progression of eosinophilic esophagitis in a subject before, during, and after treatment.

### FIELD OF THE INVENTION

This invention relates generally to the field of diagnostics. Thus, disclosed are compositions and methods for diagnosing and monitoring eosinophil degranulation-associated esophagitis in a subject, using a echogenic contrast agent administered orally to a subject. Specifically, disclosed are compositions and methods for diagnosing and monitoring eosinophilic esophagitis in a subject, using echogenic insulin aggregates linked to a targeting moiety.

### BACKGROUND

Eosinophilic esophagitis (EoE) is a chronic disease of the esophagus that affects over 300,000 patients in the U.S. alone. Symptoms include dysphagia (difficulty swallowing liquids or solids or both, >90%), food impaction (solid food sticks in the esophagus, 50%), odynophagia (painful swallowing), heartburn (33%), chest pain, asthma (50%), diarrhea, and vomiting (Gonsalves, Kahrilas, Am J Gastroenterol, 2009). The disease primarily occurs in males (75%) with a mean age between 36 and 42 years in westernized countries. While present in adults, the disease can also manifest in children. The symptoms of EoE are similar to an atopic allergenic inflammatory condition of the esophagus, affecting up to 10% of adults presenting for upper endoscopy (Mackenzie, Aliment Ther Pharmacol, Gastroenterol, 2008).

In all cases, detection of EoE via a form of endoscopy known as esophagoduodenoscopy (EGD) remains essential. In this procedure, a small tube with a camera on the distal end is passed into the esophagus, stomach, and first portion of the small intestine to visualize the mucosal surfaces of these organs. In EoE, the inflammation occurs in various parts of the esophagus; there is approximately equal incidence in the proximal, distal, or both portions of the esophagus being affected (Gangotena, Am J Gastroenterol, 2007) within cohorts, but such infiltrate varies in each individual with many demonstrating a less intense infiltrate proximally. EoE also affects the luminal structure of the esophagus. Pronounced rings or furrows can develop into strictures that close off the esophagus, resulting in odynophagia, dysphagia, food impaction, and emergency hospital visits. The areas of inflammation are not evenly distributed throughout an affected esophagus, as the disease often presents in patches or select segments of the 25-30 cm long adult esophagus.

Although EGD is a key tool in the identification of EoE, some cases may never present as a “ringed-esophagus” during EGD. A conclusive means currently available to clinicians to positively identify EoE is to detect the presence of eosinophils in biopsy specimens. Tissue samples may be collected during EGD and then examined with traditional histological analysis to confirm or reject a case of EoE. However, the patchy nature of the disease complicates collection of tissue samples for biopsy. When clinical suspicion for EoE is high, consensus practice requires sampling at 4 to 5 sites throughout the esophagus. However, five 2 mm biopsy specimens represent less than 0.7% of the 20- to 25-cm-long esophageal mucosa and might result in underdiagnosis of EoE if mucosal eosinophilia is particularly patchy. Specific disease phenotypes (i.e., rings, lines, furrows, white spots, or plaques) aid physicians in determining where and how many biopsies to perform based on EGD-observed phenotypes, which are strong indicators of eosinophil density. For example, biopsies to collect tissue samples are often collected from unaffected areas. For this reason, at least 4 (child) or 5 adult) biopsy specimens are required to confirm each case of EoE (Gonsalves Gastrointestinal Endosc, 2006; Shah Am J Gastroenterol, 2009). Furthermore, additional biopsies are required to evaluate the effectiveness of each treatment proposed. This repeated need for endoscopic removal of tissue poses a financial hardship for the patient, and the procedure can be painful, requiring sedation and/or anesthesia.

The key element for diagnosing EoE in a biopsy specimen is the presence of eosinophils. Normal esophageal tissue does not contain eosinophils (Kato et al. 1998). These white blood cells were named for their affinity for the red dye eosin. Normally, eosinophils reside in the blood stream, stomach, small and large intestine and lymphatic system (Kato et al. 1998) but infiltrate pathologically into the esophagus in EoE. In biopsy samples, an eosinophil can be identified as a cell 12-17 μm in diameter with a bilobed nucleus and cytoplasmic granules staining red with acidic dyes, for example eosin. A tissue count of eosinophils in excess of 15 per field of view at high microscope power (≧15/high-powered field (hpf)) indicates EoE. Some clinical evidence suggests that inflammation increases with eosinophil concentration.

Despite the rapidly growing incidence of EoE, state-of-the-art diagnostic techniques remain inadequate to fully characterize this disease. As such, there exists a need to develop a non-invasive, precise, and comprehensive technique to image and map the distribution of inflammation and deposition of eosinophil granule proteins. Such techniques will provide a tool to diagnose EoE, track disease activity in response to various treatment regimens, and obtain previously unreachable insight into the development and progression of EoE pathophysiology.

### SUMMARY

In accordance with the purposes of this invention, as embodied and broadly described herein, disclosed, in one aspect, are echogenic contrast agents comprising a targeting moiety linked to an echogenic amyloid aggregate such as an insulin aggregate.

In another aspect, disclosed herein are methods of diagnosing a disease in a subject comprising administering to the subject a target-specific echogenic contrast agent and detecting the presence of bound contrast agent, wherein the target-specific echogenic contrast agent binds to a target in the mucosal tissue of the subject; and wherein the presence of bound contrast agent in the mucosal tissue indicates that the subject has eosinophilic esophagitis.

In another aspect, disclosed herein are methods of producing a medical image of a tissue in a subject, comprising administering to a subject a target-specific echogenic contrast agent, wherein the target-specific echogenic contrast agent binds to the target in the mucosal tissue of the subject forming a contrast agent-target complex and detecting the presence of contrast agent-target complex in the mucosal tissue by ultrasound; and wherein detecting the echogenic contrast agent produces an image of the tissue.

In another aspect, disclosed herein are methods of monitoring the progression of a disease such as eosinophilic esophagitis in a subject diagnosed with the disease, comprising: a) producing a first medical image of the subject; b) producing a second medical image of the subject of step (a); and c) comparing the medical image of step (b) with the medical image of step (a); wherein an increase in the amount of bound contrast agent in the mucosal tissue of the subject in the medical image of step (b) relative to the medical image of step (a) indicates that the disease has worsened; and wherein a decreases in the amount of bound contrast agent in the mucosal tissue of the subject in the medical image of step (b) relative to the medical image of step (a) indicates that the disease has regressed.

### BRIEF DESCRIPTION OF THE DRAWINGS

The accompanying figures, which are incorporated in and constitute a part of this specification, illustrate several aspects and together with the description serve to explain the principles of the invention.

FIG. 1 shows histology of EoE tissue relative to normal controls. FIG. 1A shows EoE tissue histology (presence of numerous eosinophils with an affinity for the surface epithelium (numbering greater than 15 per hpf in peak areas). FIG. 1B shows normal tissue histology (no evidence of eosinophils). Fluorescently stained (1C) EoE tissue and (1D) control using antibodies to MBP-1 indicating that this granule protein is released in the disease state.

FIG. 2 shows insulin ultrasound strategy and aggregates. FIG. 2A shows a schematic view of the proposed strategy of using insulin complex to detect MBP-1 degranulation in the diseased esophagus. FIG. 2B shows Insulin aggregated incubated for 12 hours at 65° C. and pH 1.6 under the light microscopy.

FIG. 3 shows insulin ultrasound images. FIG. 3A shows an ultrasound image of insulin aggregates incubated in 15 mL centrifuge vial versus water (control). FIG. 3B shows an ultrasound image of insulin complex incubated with Macaca mulatta monkey esophagus painted with MBP-1 versus control (not treated with MBP-1) after one washing step with water.

FIG. 4 shows a transverse view of the monkey esophagus treated with MBP-1 with continuous flow of (a) water (b) insulin aggregates (c) post-washing of insulin aggregates (d) insulin complex (e) post-water washing of insulin complex.

FIG. 5 shows the thickness of insulin complex versus insulin only (control) flowing through the esophagus coated with MBP-1 as a function of time for (a) binding step (b) washing step.

FIG. 6 shows exemplary shape defined contrast agents. A 3D printer was used to generate (a) U geometries and (b) torus and 2D cross geometries used to 3D print shape defined contrast agents. Ultrasound images of (c) 8 mm letter U of panel a comprised of a PLA shell filled with suspended insulin particles, (d) 8 mm 2D cross comprised of insulin particles encapsulated within agar-agar gel, (e) 12 mm torus comprised of insulin particles encapsulated within agar-agar gel, (f), 15 mm rod comprised of insulin particles encapsulated within agar-agar gel (g) 2D cross section of insulin particle filled hollow PLA toroid. These contrast agents can be distinguished from local noise generated by bubble rich liquid and reverberations from gel/liquid, solid/liquid, and air/liquid interfaces. Each tick mark on the right axis spans 0.5 cm.

FIG. 7 shows that the insulin particles can be readily imaged at water-soft tissue-like interfaces. The interface is denoted by a white dashed line. (a) Insulin particles in both water and tissue simulant of pudding-like consistency. (b) Insulin particles in tissue simulant of pudding-like consistency but not in the water. Triangles on right axis span 1 cm.

FIG. 8 shows (a) small insulin particles sieved to approximately 50-75 microns, (b) medium insulin particles sieved to approximately 75-100 microns, and (c) large insulin particles sieved to approximately >100 microns. (d) The insulin particles capture instantaneous flow profiles in solution. Tick marks on right axis span 0.5 cm.

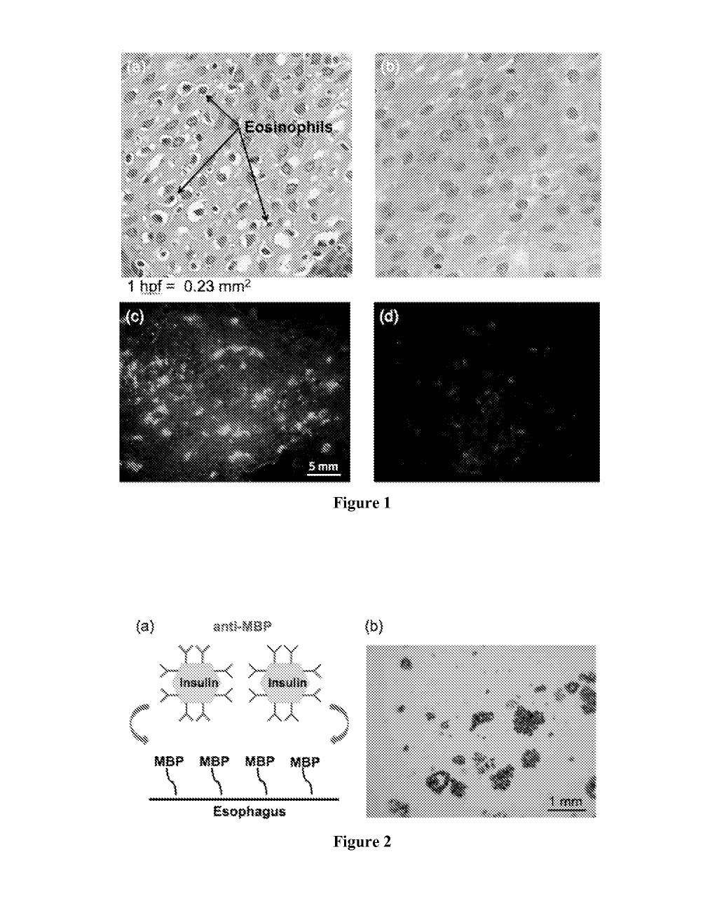

### DETAILED DESCRIPTION

What is needed in the art are compositions and non-invasive methods for diagnosing disease (e.g., eosinophil esophagitis) or imaging tissue in a subject and for monitoring the effectiveness of treatment in the subject in order to decrease suffering and cost and to increase subject compliance. Eosinophil esophagitis degranulation-associated esophagitis is eosinophilic esophagitis (EoE). The disclosed compositions are designed to bind a target moiety and be visualized via ultrasound or MRI. The compositions can be used in methods to diagnose eosinophilic esophagitis in a subject by detecting the presence of eosinophil granule proteins in the esophageal mucosal tissue. Thus, the diagnosis can be made even when morphologically intact eosinophils cannot be found in the inflamed tissue under microscopic examination.

The present invention may be understood more readily by reference to the following detailed description of various aspects of the invention and the Examples included herein and to the Figures and their previous and following description.

Before the present compounds, compositions, articles, devices, and/or methods are disclosed and described, it is to be understood that this invention is not limited to specific synthetic methods or specific echogenic contrast agents, as such may, of course, vary. It is also to be understood that the terminology used herein is for the purpose of describing particular aspects only and is not intended to be limiting.

As used in the specification and the appended claims, the singular forms “a,” “an,” and “the” include plural referents unless the context clearly dictates otherwise. Thus, for example, reference to “a echogenic contrast agent” or a “echogenic contrast agent/eosinophil granule protein complex” can include mixtures of echogenic contrast agents or mixtures of echogenic contrast agent/eosinophil granule protein complexes, respectively, and the like.

Ranges may be expressed herein as from “about” one particular value and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant, both in relation to the other endpoint and independently of the other endpoint.

As used herein “subject” refers to a mammal receiving the compositions disclosed herein or subject to the disclosed methods. It is understood and herein contemplated that “mammal” includes but is not limited to humans, non-human primates, cows, horses, dogs, cats, mice, rats, rabbits, and guinea pigs.

### Compositions

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a echogenic contrast agent is disclosed and discussed and a number of modifications that can be made to a number of molecules including the echogenic contrast agent, amyloid aggregate, and targeting moiety are discussed, each and every combination and permutation of the echogenic contrast agent, amyloid aggregate, and targeting moiety and the modifications that are possible are specifically contemplated unless specifically indicated to the contrary. Thus, if a class of molecules A, B, and C is disclosed as well as a class of molecules D, E, and F and an example of a combination molecule A-D is disclosed, then even if each is not individually recited, each is individually and collectively contemplated. Thus, in this example, each of the combinations A-E, A-F, B-D, B-E, B-F, C-D, C-E, and C-F is specifically contemplated and should be considered disclosed from disclosure of A, B, and C; D, E, and F; and the example combination A-D. Likewise, any subset or combination of these is also specifically contemplated and disclosed. Thus, for example, the sub-groups of A-E, B-F, and C-E are specifically contemplated and should be considered disclosed from disclosure of A, B, and C; D, E, and F; and the example combination A-D. This concept applies to all aspects of this application including, but not limited to, steps in the methods of making and using the disclosed echogenic contrast agents and amyloid aggregate-heparin/eosinophil granule protein complexes and amyloid aggregate-antibody/eosinophil granule protein complexes. Thus, if there are a variety of additional steps that can be performed, it is understood that each of these additional steps can be performed with any specific aspect or combination of aspects of the disclosed methods and that each such combination is specifically contemplated and should be considered disclosed.

Those skilled in the art will recognize, or be able to ascertain using no more than routine experimentation, many equivalents to the specific aspects of the methods and compositions described herein. Such equivalents are intended to be encompassed by the appended claims.

Unless defined otherwise, all technical and scientific terms used herein have the same meanings as commonly understood by one of skill in the art to which the disclosed methods and compositions belong. Although any methods and materials similar or equivalent to those described herein can be used in the practice or testing of the disclosed methods and compositions, the particularly useful methods, devices, and materials are as described.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents.

It is understood that the disclosed methods and compositions are not limited to the particular methodology, protocols, and reagents described, as these may vary. It is also to be understood that the terminology used herein is for the purpose of describing particular aspects only and is not intended to limit the scope of the present invention which will be limited only by the appended claims.

In this specification and in the claims which follow, reference will be made to a number of terms which shall be defined to have the following meanings. The word “comprise” and variations of the word, such as “comprising” and “comprises,” means “including, but not limited to” and is not intended to exclude, for example, other additives, components, integers or steps. “Optional” or “optionally” means that the subsequently described event or circumstance may or may not occur and that the description includes instances where said event or circumstance occurs and instances where it does not. As used herein, by “subject” is meant an individual. A subject can be a mammal such as a primate, for example, a human. The term “subject” includes domesticated animals such as cats, dogs, etc., livestock (e.g., cattle, horses, pigs, sheep, goats, etc.), and laboratory animals (e.g., mice, rabbits, rats, gerbils, guinea pigs, possums, etc.). As used herein, the terms “subject” and “patient” are interchangeable.

Ultrasound is a powerful imaging and diagnostic tool for bulk tissues because it is noninvasive, safe, rapid and even quantitative. However, most disease processes occur at the cellular or subcellular level beyond ultrasound resolution. Moreover, although traditional ultrasound contrast agents such as microbubbles are bright, they are not useful in the lungs and luminal cavities (e.g. esophagi) because of their limited contrast near air-water-tissue surfaces, their rapid dissolution, and the creation of nonspecific air bubbles due to turbulent peristalsis in vivo. Thus, microbubbles are less effective for diagnosing diseases that are present in the luminal, such as, for example, eosinophilic esophagitis. New contrast agents are needed to provide contrast in multiphase (tissue, viscoelastic mucous, aqueous, and air) environments. Disclosed herein are novel echogenic contrast agents developed so that ultrasound can access biomolecular densities.

Leukocyte activation is a critical step in many immunological diseases affecting millions of Americans, including asthma and eosinophilic esophagitis (EoE). In EoE, eosinophils infiltrate the esophagus (normally devoid thereof), where they activate, degranulate and release an array of highly basic, cytotoxic proteins including major basic protein (MBP-1). During activation, MBP-1 accumulates on the surface of eosinophil membranes prior to degranulation and release steps. The present disclosure provides for, amongst other things, a new class ultrasound contrast agents to determine MBP-1 concentration in the esophagus as a marker of eosinophil activation, infiltration, and disease.

As used herein, a “mucosal tissue” is a tissue lining various cavities within the body. Examples of a mucosal tissue include, but are not limited to, mucosal tissue lining the nose, sinuses, bronchi, lungs, conjunctiva, oral cavity, tongue, esophagus, stomach, pylorus, duodenum, jejunum, ileum, ascending colon, caecum, appendix, transverse colon, descending colon, rectum, anus, urethra, and urinary bladder. A mucosal tissue comprises an epithelial surface, glandular epithelium which secretes mucus, basement membrane, and submucosa with connective tissue. Thus, a echogenic contrast agent-heparin/eosinophil granule protein complex or echogenic contrast agent-antibody/eosinophil granule protein complex can be detected on the epithelial surface, in the glandular epithelial tissue, and in the submucosa connective tissue of a mucosal tissue in a subject. In one aspect, a mucosal tissue is from the esophagus of a subject.

As used herein, an “eosinophil granule protein” is a protein that comprises the granules in eosinophils. When an eosinophil is activated, granule proteins are released from the cell into the surrounding tissue. The released granule proteins can cause pathologic allergenic inflammatory responses in the surrounding tissue, for example esophageal mucosal tissue. Examples of eosinophil granule proteins include, but are not limited to, major basic protein (MBP), major basic protein 1 (MBP-1), major basic protein 2 (MBP-2), eosinophil derived neurotoxin (EDN), eosinophil cationic protein (ECP), and eosinophil peroxidase (EPO). Other examples of eosinophil granule proteins are provided in Kita et al., Biology of Eosinophils, Chapter 19 of Immunology, which is hereby incorporated by reference for its teaching of examples of eosinophil granule proteins. In one aspect, an eosinophil granule protein can be MBP-1.

In one aspect, the disclosed echogenic contrast agents comprise an amyloid aggregate (such as, for example, an insulin aggregate) and a targeting moiety. In other aspects the contrast agent does not require a targeting moiety and comprises the amyloid aggregate alone. Thus, in another aspect, disclosed herein are echogenic contrast agents comprising an amyloid aggregate, such as an insulin aggregate.

### Amyloid Aggregates

It is understood and herein contemplated that amyloid fibrils, such as, for example, insulin provide advantageous properties that make them suitable for use in the echogenic contrast agent. Insulin was selected as an example of an echogenic amyloid fibril because it forms amyloid fibrils at low pH and elevated temperature, and is readily digested by gastric acid in the stomach minimizing the risk of amyloidosis. Insulin fibrils provide distinct contrast because of their unusual compliance (partial specific compressibility is −0.9*10-6 mL/g*bar) and harmonic resonance. Accordingly, in one aspect, disclosed herein are echogenic contrast agents comprising an targeting moiety or heparin linked to an echogenic amyloid fibril, wherein the amyloid fibril is an insulin aggregate.

In one aspect, it is contemplated that the size of the aggregate can be varied depending on the application. Thus, in one aspect, disclosed herein are amyloid aggregates such as insulin aggregates wherein the aggregates are at least 10, 15, 20, 25, 30, 35, 45, 45, 50, 60, 70, 80, 90, 100, 200, 300, 400, 500, 600, 700, 800, 900 nanometers, 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 100, 125, 150, 175, 200, 250, 300, 350, 400, 450, 500, 600, 700, 800, 900, 1000 microns, 2, 3, 4, 5, 6, 7, 8, 9 10, 11, 12, 13, 14, 15, 16, 17, 18, 19, 20, 25, 30, 35, 40, 45, or 50 mm in size or any size in between. For example the insulin aggregate can be between about 25 and about 100 microns. In one aspect, the insulin aggregates can be sieved to separate the aggregates by size. For example, small insulin aggregates can be about 50-75 microns, medium insulin aggregates can be about 75-100 microns, and large insulin aggregates can be about 100 microns or larger.

In one aspect, it is contemplated that echogenic aggregated amyloid fibrils such as insulin can be allowed to form randomly shaped aggregate structures or formed into specific shapes. For example, it is contemplated herein that the amyloid aggregates, such as insulin aggregates, can be formed in to a cylindrical, circular, rod, rectangular, star, cross, ellipsoidal, smiley face, frowney face, skull and cross-bones, letter, or any other shape desired. Thus, disclosed herein in one aspect are echogenic contrast agents wherein the echogenic amyloid aggregate is insulin, and wherein the insulin aggregate has a cylindrical, circular, rod, rectangular, star, cross, ellipsoidal, smiley face, frowney face, skull and cross-bones, or letter shape Three dimensional shapes can be used so that rotation through the imaging plane is not an issue. These 3D shapes include but are not limited to 3D letters such as 3D U's, cubes, 3D rectangles or cuboids, tetrahedra, 3D crosses or pluses, 3D crossed crosses, crosses with looped elements, dodecahedra, icosahedra, octahedra, truncated octahedra, cuboctahedra, great rhombicosidodecahedra, great rhombicuboctahedra, icosidodecahedra, small rhombicosidodecahedra, small rhombicuboctahedra, snub cubes, snub dodecahedra, truncated cubes, truncated dodecahedra, truncated icosahedra, truncated octahedra, and truncated tetrahedra. The 3D shapes also include a wide variety of other concave, convex, and asymmetric structures which include but are not limited to crescent moons with triangular cross sections with or without curved edges. Structures that are internally empty such as 3D rectangles and tetrahedra with toroidal edges are can also be used. It is further understood that there are instances where the use of multiple echogenic contrast agents targeting different determinants in the tissue of a subject is desired. In such instances, it is contemplated herein that the echogenic amyloid aggregate (e.g., insulin) can be formed into different shapes for each targeting moiety to distinguish contrast agent-target complexes. Such shapes can be made utilizing a 3D biomaterial printer to make image molds. The molds can be made of polyvinyl alcohol or other water soluble polymer facilitating release of the injected amyloid fibril. For example, poly(dimethyl siloxane) (PDMS), silastic 4210 from Dow Corning. acrylonitrile butadiene styrene (ABS), poly(vinyl alcohol) (PVA), polylactide, polyglycolide, poly(lactide co-glycolide), polycaprolactone, and any combination thereof can be used to print the molds. Once the forms are made, insulin can be injected directly into the form and holes from inject filled with a biocompatible glue such as cyanoacrylate. Alternatively, a gel can be injected into the mold and the amyloid fibril such as insulin can be injected within the gel removing the need for a cap. As another alternative, the amyloid fibril, such as insulin, can be injected into the mold within a gelling solution. The gelled insulin is then released from the mold. It is understood and herein contemplated that the mold can be a water soluble polymer shell and can be dissolved to expose the internal structure within it.

The gels can comprise lower elastic moduli ≦100 MPa. Softer materials are advantageous because they provide less “screening” of the acoustic waves. Materials that are too soft (e.g., 1 kPa) may deform too much for the shapes to be recognized. Accordingly, disclosed herein are gelling materials with a modulus between about 10 kPa and about 10 MPa, between about 20 kPa and about 5 MPa, between about 50 kPa and about 1 MPa, and between about 100 kPa and about 1 MPa. For example, the gelling material can be at least 10, 11, 12, 13, 14, 15, 16, 17, 18, 19, 20, 25, 30, 35, 40, 45, 50, 55, 60, 65, 70, 75, 80, 85, 90, 100, 110, 120, 130, 140, 150, 160, 170, 180, 190, 200, 250, 300, 350, 400, 450, 500, 600, 700, 800, 900 kPa, 1, 2, 3, 4, 5, 6, 7, 8, 9, 10, 15, 20, 25, 30, 35, 40, 45, 50, 55, 60, 65, 70, 75, 80, 85, 90, 95 or 100 MPa.

Gelling material can be made of any biocompatible polymer or co-polymer. For example, the gelling material can comprise agar-agar, alginate, carrageenan, cassia gum, cellulose gum, gellan gum, guar gum, hydroxypropylcellulose, konjac gum, locust bean gum, methylcellulose and hydroxypropyl methylcellulose, microcrystalline cellulose, pectin, xanthan gum, polylactide, polyglycolide, poly(lactide co-glycolide), polycaprolactone, or any combination thereof.

In another aspect, the disclosed amyloid aggregate can further comprise USP quality minerals such as, for example, zinc, cobalt as vitamin B₁₂, iron, magnesium, and potassium. The minerals can be dissolved in water and mixed with the insulin. As the particles form, the metals incorporate in the particle. The addition of the mineral provides a distinctive MRI signature which allows the ability for the differentiation of different targets.

### Targeting Moieties

In one aspect, it is understood and herein contemplated that the echogenic contrast agents disclosed herein can be targeted to a particular protein, peptide, or other antigenic determinant. For example, the targeting moiety can be anionic heparin, polyglutamic acid, polyaspartic acid, or an antibody specific for a protein, peptide, or antigenic determinant such as an antibody specific for a eosinophil protein such as a eosinophil granule protein. Eosinophilic granule proteins can comprise any such protein, including but not limited to major basic protein 1 (MBP-1), major basic protein 2 (MBP-2), eosinophil derived neurotoxin (EDN), eosinophil cationic protein (ECP), or eosinophil peroxidase (EPO). Thus, in one aspect, disclosed herein are echogenic contrast agents comprising an amyloid aggregate such as an insulin aggregate linked to an antibody specific for an eosinophil granular protein, wherein the eosinophil granule protein comprises MBP-1, MBP-2, EDN, ECP, or EPO.

Antibodies

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (K) and lambda (4 based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse, rat, guinea pig or other mammals. The heavy chain constant domains that correspond to the different classes of immunoglobulins are called alpha, delta, epsilon, gamma, and mu, respectively.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a β-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

As used herein, the term “antibody or fragments thereof” encompasses chimeric antibodies and hybrid antibodies, with dual or multiple antigen or epitope specificities, and fragments, such as F(ab′)2, Fab′, Fab, Fv, sFv, and the like, including hybrid fragments. Thus, fragments of the antibodies that retain the ability to bind their specific antigens are provided. For example, fragments of antibodies which maintain target binding activity are included within the meaning of the term “antibody or fragment thereof” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

Optionally, the antibodies are generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296 (1993) and Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding (see, WO 94/04679, published 3 Mar. 1994).

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA, 90:2551-255 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boerner et al. are also available for the preparation of human monoclonal antibodies (Cole et al., Monoclonal Antibodies and Cancer Therapy, Alan R. Liss, p. 77 (1985); Boerner et al., J. Immunol., 147(1):86-95 (1991)).

Disclosed are hybridoma cells that produce the monoclonal antibody. The term “monoclonal antibody” as used herein refers to an antibody obtained from a substantially homogeneous population of antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired activity (See, U.S. Pat. No. 4,816,567 and Morrison et al., Proc. Natl. Acad. Sci. USA, 81:6851-6855 (1984)).

Monoclonal antibodies may be prepared using hybridoma methods, such as those described by Kohler and Milstein, Nature, 256:495 (1975) or Harlow and Lane. Antibodies, A Laboratory Manual. Cold Spring Harbor Publications, New York, (1988). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, guinea pigs, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

It is understood and herein contemplated that the amyloid aggregate can be directly or indirectly linked to the targeting moiety. Examples of linking systems include but are not limited to biotin-avidin, copper-catalyzed azide alkyne cylcoaddition, and succinimidyl-diazirine (SDA). For example, in one aspect, the targeting moiety and the amyloid aggregate such as an insulin aggregate can be linked via a biotin-avidin interaction. Alternatively or in addition, a linking system can comprise NHS-esters to target lysine residues and/or maleimides to target cystein residues on the targeting moiety and amyloid aggregate. It is understood and herein contemplated that the a lining system could comprise both NHS-esters and maleimides at opposing ends of the linker or NHS-esters or maleimides at both ends. Thus, in one aspect, disclosed herein are biotinylated antibodies. Also disclosed herein are amyloid aggregates such as insulin aggregates wherein the amyloid aggregate is conjugated with an avidin such as streptavidin. In another aspect, the echogenic contrast agent comprises an insulin aggregate with a bound succinimidyl moiety and heparin, wherein heparin is reacted with a diazirine moiety.

### Methods

The echogenic contrast agent compositions disclosed herein can be used for non-invasive methods to visualize tissues that otherwise would require more invasive techniques to visualize. Due to the ability to visualize tissue, the disclosed echogenic contrast agents can be used to diagnose a disease or condition or to monitor the progression of a disease or condition.

In one aspect, disclosed herein are methods of producing a medical image of a tissue in a subject, comprising administering to a subject an target-specific echogenic contrast agent, wherein the target-specific echogenic contrast agent binds to the target in the mucosal tissue of the subject forming a contrast agent-target complex and detecting the presence of contrast agent-target complex in the mucosal tissue by ultrasound; and wherein detecting the echogenic contrast agent produces an image of the tissue. For example disclosed herein are methods of producing a medical image of an esophagus in a subject, comprising a) administering to a subject an eosinophil protein-specific echogenic contrast agent, wherein the eosinophil protein-specific echogenic contrast agent binds to a eosinophil protein in the mucosal tissue of the esophagus forming a contrast agent-eosinophil protein complex and b) detecting the presence of contrast agent-eosinophil protein complex in the mucosal tissue of the esophagus by ultrasound; and wherein detecting the echogenic contrast agent produces an image of the esophagus.

After administering to a subject a composition comprising an echogenic contrast agent, for example insulin aggregate-anti-MBP1, insulin aggregate-anti-MBP2, insulin aggregate-anti-EPO, insulin aggregate-anti-EDN, insulin aggregate-anti-ECP, a person of ordinary skill can use one or more technologies and processes to detect amyloid aggregate-heparin/eosinophil granule protein complexes, amyloid aggregate-anti-MP1/eosinophil granule protein complexes, amyloid aggregate-anti-MBP2/eosinophil granule protein complexes, amyloid aggregate-anti-EPO/eosinophil granule protein complexes, amyloid aggregate-anti-EDN/eosinophil granule protein complexes, amyloid aggregate-anti-ECP/eosinophil granule protein complexes in the mucosal tissue of the esophagus in a subject, where eosinophils have degranulated and caused one or more patches of inflammation, to create a medical image to map the distribution of inflammation and deposition of eosinophil granule proteins to study the anatomy and/or pathophysiology of eosinophilic esophagitis. Examples of technologies that can be used to create a medical image include, but are not limited to ultrasound or magnetic resonance imaging (MRI). In one aspect, for example, SPECT can optionally be used in combination with MRI and/or CT scans to produce a medical image of an esophagus having patches of eosinophilic esophagitis. Fiduciary markers on the skin of a subject can also be used to position a subject so that the subject can be imaged from day to day. For example, lasers can be used to position a subject reproducibly. This permits use of multiple scans to be precisely compared. In one aspect, a medical image can be three-dimensional. In another aspect, a medical image can be two-dimensional.

In one aspect, an echogenic contrast agent, for example an echogenic insulin aggregate-heparin or echogenic insulin aggregate-antibody (e.g., insulin aggregate-anti-MBP-1), can be administered to a subject orally. Oral dosing can entail ingestion similar to routine barium studies of the esophagus. A echogenic contrast agent can be suspended in a thickened mixture (i.e., sucralose). Examples of thickening agents include, but are not limited to, dietary starches, such as agar-agar, alginate, carrageenan, cassia gum, cellulose gum, gellan gum, guar gum, hydroxypropylcellulose, konjac gum, locust bean gum, methylcellulose, hydroxypropyl methylcellulose, microcrystalline cellulose, pectin, and xanthan gum. Other viscosifiers include honey, agave nectar, date nectar, Kuzu, arrow root, corn syrup, thick juices, maple syrup, coconut oil, palm oil, polylactide, polyglycolide, poly(lactide co-glycolide), polycaprolactone, and any combination thereof.

The dwell time in the esophagus can be controlled by varying the viscosity of a contrast agent and by increasing the time interval between swallows, thereby providing more time for a contrast agent to contact and bind to an eosinophil granule protein. Further, having a subject lie down with head below feet so that there is some reflux within the esophagus can prolong contact between a contrast agent and the mucosal tissue of the esophagus in a subject.

A echogenic contrast agent can be administered to a subject in a volume from about 0.5 mL to about 500 mL, including all volumes in between 0.5 mL and 500 mL. A person of ordinary skill can determine by methods well known in the art the volume of a contrast agent to be administered to a subject based on the age, sex, weight, and overall condition of a subject. For example, in one aspect, the volume of a contrast agent administered to a subject can be from about 5 mL to about 250 mL. In another aspect, the volume of a contrast agent administered to a subject can be from about 10 mL to about 125 mL. In another aspect, the volume of a contrast agent administered to a subject can be from about 15 mL to about 50 mL. Thus, the volume of a contrast agent that can be administered to a subject can be, for example, about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mL, and all volumes in between.

It is understood and herein contemplated that other routes of administration of the echogenic contrast agent can be utilized including but limited to aerosol administration, enema, and intraurethral administration.

In another aspect disclosed herein are methods of diagnosing a disease such as for example, allergies (including food allergies); circulatory blockage; bowel motility; urinary blockage; pelvic floor disorders, colitis; gastritis; eosinophilic esophagitis; eosinophilic gastroenteritis; eosinophil-associated bladder diseases; biliary tract disease (including but not limited to primary sclerosing cholangitis); bacterial/mycotic/parasitic diseases of the gastrointestinal tract including but not limited to Giardia, Salmonella; Parasitic infection, Shigella, Campylobacter, E. coli O157:H7, Cyclospora, Cryptosporidium, and C. difficile; and bacterial/mycotic/parasitic diseases of the bladder and urinary tract E. coli, Pseudomonas, enterococcus, group B strep, Klebsiella, Staph saprophyticus, Enterobacter sp, and Proteus sp. Thus, disclosed herein in one aspect are methods of diagnosing a disease comprising administering to a subject one or more of the echogenic contrast agents disclosed herein, wherein the presence of bound contrast agent in a target tissue indicates that the subject has the disease. For example, disclosed herein are methods of diagnosing eosinophilic esophagitis in a subject, comprising administering to a subject an eosinophil protein-specific echogenic contrast agent and detecting the presence of bound contrast agent in the mucosal tissue of the esophagus, wherein the eosinophil protein-specific echogenic contrast agent binds to a eosinophil protein in the mucosal tissue of the esophagus; and wherein the presence of bound contrast agent in the mucosal tissue of the esophagus indicates that the subject has eosinophilic esophagitis.

After administering to a subject a composition comprising an echogenic contrast agent, for example insulin aggregate-anti-MBP1, insulin aggregate-anti-MBP2, insulin aggregate-anti-EPO, insulin aggregate-anti-EDN, insulin aggregate-anti-ECP, a person of ordinary skill can use one or more technologies and processes to detect amyloid aggregate-heparin/eosinophil granule protein complexes, amyloid aggregate-anti-MBP1/eosinophil granule protein complexes, amyloid aggregate-anti-MBP2/eosinophil granule protein complexes, amyloid aggregate-anti-EPO/eosinophil granule protein complexes, amyloid aggregate-anti-EDN/eosinophil granule protein complexes, amyloid aggregate-anti-ECP/eosinophil granule protein complexes in the mucosal tissue of the esophagus in a subject, where eosinophils have degranulated and caused one or more patches of inflammation, to create a medical image to map the distribution of inflammation and deposition of eosinophil granule proteins to study the anatomy and/or pathophysiology of eosinophilic esophagitis. Examples of technologies that can be used to create a medical image include, but are not limited to ultrasound or magnetic resonance imaging (MRI). In one aspect, for example, SPECT can optionally be used in combination with MRI and/or CT scans to produce a medical image of an esophagus having patches of eosinophilic esophagitis. Fiduciary markers on the skin of a subject can also be used to position a subject so that the subject can be imaged from day to day. For example, lasers can be used to position a subject reproducibly. This permits use of multiple scans to be precisely compared. In one aspect, a medical image can be three-dimensional. In another aspect, a medical image can be two-dimensional.

In one aspect, a echogenic contrast agent, for example echogenic insulin aggregate-heparin or echogenic insulin aggregate-antibody (e.g., insulin aggregate-anti-MBP-1), can be administered to a subject orally. Oral dosing can entail ingestion similar to routine barium studies of the esophagus. A echogenic contrast agent can be suspended in a thickened mixture (i.e., sucralose). Examples of thickening agents include, but are not limited to, dietary starches, such as agar-agar, alginate, carrageenan, cassia gum, cellulose gum, gellan gum, guar gum, hydroxypropylcellulose, konjac gum, locust bean gum, methylcellulose, hydroxypropyl methylcellulose, microcrystalline cellulose, pectin, and xanthan gum. Other viscosifiers include honey, agave nectar, date nectar, Kuzu, arrow root, corn syrup, thick juices, maple syrup, coconut oil, palm oil, polylactide, polyglycolide, poly(lactide co-glycolide), polycaprolactone, and any combination thereof.

The dwell time in the esophagus can be controlled by varying the viscosity of a contrast agent and by increasing the time interval between swallows, thereby providing more time for a contrast agent to contact and bind to an eosinophil granule protein. Further, having a subject lie down with head below feet so that there is some reflux within the esophagus can prolong contact between a contrast agent and the mucosal tissue of the esophagus in a subject.

A echogenic contrast agent can be administered to a subject in a volume from about 0.5 mL to about 500 mL, including all volumes in between 0.5 mL and 500 mL. A person of skill can determine by methods well known in the art the volume of a contrast agent to be administered to a subject based on the age, sex, weight, and overall condition of a subject. For example, in one aspect, the volume of a contrast agent administered to a subject can be from about 5 mL to about 250 mL. In another aspect, the volume of a contrast agent administered to a subject can be from about 10 mL to about 125 mL. In another aspect, the volume of a contrast agent administered to a subject can be from about 15 mL to about 50 mL. Thus, the volume of a contrast agent that can be administered to a subject can be, for example, about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mL, and all volumes in between.

It is understood and herein contemplated that other routes of administration of the echogenic contrast agent can be utilized including but not limited to aerosol administration, enema, and intraurethral administration.

In another aspect, disclosed herein are methods of diagnosing an allergy (including environmental and food allergies) in a subject, comprising inducing an allergic reaction by exposure to the relevant allergen and detecting the consequences of the induced inflammation with the understanding that this inflammation is enriched in eosinophil granule proteins. The granule proteins can be detected using the procedures described earlier in this document and including MBP1, MBP2, EDN, ECP and EPO. Alternatively, the methods of diagnosing an allergies can comprise imaging techniques analyzing tissues for eosinophilic infiltrates upon prolonged (24-48) or immediate (1 minutes to 12 hours) exposure to the antigen/allergen comprising detection of eosinophil granule proteins including EDN, EPO, and MBP within tissues relatively devoid of such proteins in the normal state. Tissues for evaluating in this manner include but are not limited to nasopharangeal, bronchial, bladder, large and small intestine, esophagus, vascular, biliary.

In another aspect, the disclosed echogenic contrast agents can be used to detect circulatory or urinary blockage or to track bowel motility. In such methods, the contrast agent does not comprise heparin or an antibody but comprises the aggregate of the amyloid fibril (for example, insulin aggregates). The contrast agent is administered to the subject and traced using ultrasound or MRI; where there is an accumulation of echogenic contrast agent, there is a blockage.

In one aspect disclosed herein are methods of monitoring the progression of eosinophilic esophagitis in a subject diagnosed with eosinophilic esophagitis, comprising: a) producing a first medical image of the esophagus of the subject according to the imaging methods disclosed herein; b) producing a second medical image of the esophagus in the subject of step (a) according to the imaging methods disclosed herein; and c) comparing the medical image of step (b) with the medical image of step (a); wherein an increase in the amount of bound contrast agent in the mucosal tissue of the esophagus in the medical image of step (b) relative to the medical image of step (a) indicates that the eosinophilic esophagitis has worsened; and wherein a decreases in the amount of bound contrast agent in the mucosal tissue of the esophagus in the medical image of step (b) relative to the medical image of step (a) indicates that the eosinophilic esophagitis has regressed.

After administering to a subject a composition comprising echogenic contrast agent, for example insulin aggregate-anti-MBP1, insulin aggregate-anti-MBP2, insulin aggregate-anti-EPO, insulin aggregate-anti-EDN, insulin aggregate-anti-ECP, a person of ordinary skill can use one or more technologies and processes to detect amyloid aggregate-heparin/eosinophil granule protein complexes, amyloid aggregate-anti-MP1/eosinophil granule protein complexes, amyloid aggregate-anti-MBP2/eosinophil granule protein complexes, amyloid aggregate-anti-EPO/eosinophil granule protein complexes, amyloid aggregate-anti-EDN/eosinophil granule protein complexes, amyloid aggregate-anti-ECP/eosinophil granule protein complexes in the mucosal tissue of the esophagus in a subject, where eosinophils have degranulated and caused one or more patches of inflammation, to create a medical image to map the distribution of inflammation and deposition of eosinophil granule proteins to study the anatomy and/or pathophysiology of eosinophilic esophagitis. Examples of technologies that can be used to create a medical image include, but are not limited to ultrasound or magnetic resonance imaging (MRI). In one aspect, for example, SPECT can optionally be used in combination with MRI and/or CT scans to produce a medical image of an esophagus having patches of eosinophilic esophagitis. Fiduciary markers on the skin of a subject can also be used to position a subject so that the subject can be imaged from day to day. For example, lasers can be used to position a subject reproducibly. This permits use of multiple scans to be precisely compared. In one aspect, a medical image can be three-dimensional. In another aspect, a medical image can be two-dimensional.

In one aspect, an echogenic contrast agent, for example echogenic insulin aggregate-heparin or echogenic insulin aggregate-antibody (e.g., insulin aggregate-anti-MBP-1), can be administered to a subject orally. Oral dosing can entail ingestion similar to routine barium studies of the esophagus. A echogenic contrast agent can be suspended in a thickened mixture (i.e., sucralose). Examples of thickening agents include, but are not limited to, dietary starches, such as agar-agar, alginate, carrageenan, cassia gum, cellulose gum, gellan gum, guar gum, hydroxypropylcellulose, konjac gum, locust bean gum, methylcellulose, hydroxypropyl methylcellulose, microcrystalline cellulose, pectin, and xanthan gum. Other viscosifiers include honey, agave nectar, date nectar, Kuzu, arrow root, corn syrup, thick juices, maple syrup, coconut oil, palm oil, polylactide, polyglycolide, poly(lactide co-glycolide), polycaprolactone, and any combination thereof.

The dwell time in the esophagus can be controlled by varying the viscosity of a contrast agent and by increasing the time interval between swallows, thereby providing more time for a contrast agent to contact and bind to an eosinophil granule protein. Further, having a subject lie down with head below feet so that there is some reflux within the esophagus can prolong contact between a contrast agent and the mucosal tissue of the esophagus in a subject.

An echogenic contrast agent can be administered to a subject in a volume from about 0.5 mL to about 500 mL, including all volumes in between 0.5 mL and 500 mL. A person of skill can determine by methods well known in the art the volume of a contrast agent to be administered to a subject based on the age, sex, weight, and overall condition of a subject. For example, in one aspect, the volume of a contrast agent administered to a subject can be from about 5 mL to about 250 mL. In another aspect, the volume of a contrast agent administered to a subject can be from about 10 mL to about 125 mL. In another aspect, the volume of a contrast agent administered to a subject can be from about 15 mL to about 50 mL. Thus, the volume of a contrast agent that can be administered to a subject can be, for example, about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mL, and all volumes in between.

It is understood and herein contemplated that other routes of administration of the echogenic contrast agent can be utilized including but limited to aerosol administration, enema, and intraurethral administration.

Disclosed herein are echogenic contrast agents comprising an targeting moiety linked to an echogenic insulin aggregate.

In another aspect disclosed herein is any preceding echogenic contrast agent, wherein the targeting moiety is specific for or binds to an eosinophilic peptide or protein present in the mucosal tissue surrounding an eosinophil.

Also disclosed are echogenic contrast agents of any preceding aspect, wherein the eosinophilic peptide or protein comprises major basic protein 1 (MBP-1), major basic protein 2 (MBP-2), eosinophil derived neurotoxin (EDN), eosinophil cationic protein (ECP), or eosinophil peroxidase (EPO).

Also disclosed are echogenic contrast agents of any preceding aspect, wherein the targeting moiety is an antibody, polyglutamic acid, polyaspartic acid, or an anionic heparin. While polyglutamic acid and polyaspeartic acid are specifically named, it is understood, that any polyanion can work.

Also disclosed are echogenic contrast agents of any preceding aspect, wherein the targeting moiety is biotinylated.

Also disclosed are echogenic contrast agents of any preceding aspect, wherein the echogenic insulin aggregate is conjugated with streptavidin.

Also disclosed are echogenic contrast agents of any preceding aspect wherein the insulin aggregate has a cylindrical, circular, star, cross, rod, rectangular, smiley face, frowny face, or ellipsoidal shape. Three dimensional shapes can be used so that rotation through the imaging plane is not an issue. These 3D shapes include but are not limited to 3D letters such as 3D U's, cubes, 3D rectangles or cuboids, tetrahedra, 3D crosses or pluses, 3D crossed crosses, crosses with looped elements, dodecahedra, icosahedra, octahedra, truncated octahedra, cuboctahedra, great rhombicosidodecahedra, great rhombicuboctahedra, icosidodecahedra, small rhombicosidodecahedra, small rhombicuboctahedra, snub cubes, snub dodecahedra, truncated cubes, truncated dodecahedra, truncated icosahedra, truncated octahedra, and truncated tetrahedra. The 3D shapes also include a wide variety of other concave, convex, and asymmetric structures which include but are not limited to crescent moons with triangular cross sections with or without curved edges. Structures that are internally empty such as 3D rectangles and tetrahedra with toroidal edges are can also be used

In another aspect, disclosed herein are methods of diagnosing eosinophilic esophagitis in a subject, comprising administering to a subject an eosinophil protein-specific echogenic contrast agent and detecting the presence of bound contrast agent in the mucosal tissue of the esophagus, wherein the eosinophil protein-specific echogenic contrast agent binds to a eosinophil protein in the mucosal tissue of the esophagus; and wherein the presence of bound contrast agent in the mucosal tissue of the esophagus indicates that the subject has eosinophilic esophagitis.

Also disclosed are any preceding method, wherein the eosinophil protein-specific echogenic contrast agent comprises an echogenic insulin aggregate linked to a targeting moiety specific for or that binds to an eosinophil protein.

Also disclosed are any preceding method, wherein the eosinophil protein is an eosinophil granule protein.

Also disclosed are any preceding method, wherein the eosinophil granule protein comprises MBP-1, MBP-2, EDN, ECP, or EPO.

Also disclosed are any preceding method, wherein the eosinophil granule protein is MBP-1.

Also disclosed are any preceding method, wherein the eosinophil protein specific targeting moiety is linked to the echogenic insulin aggregate via a biotin-streptavidin interaction, wherein the targeting moiety is biotinylated and the insulin aggregate conjugated with streptavidin.

Also disclosed are any preceding method, wherein the targeting moiety is an antibody, polyglutamic acid, polyaspartic acid, or heparin. While polyglutamic acid and polyaspeartic acid are specifically named, it is understood, that any polyanion can work.

Also disclosed are any preceding method, wherein the eosinophil protein-specific echogenic contrast agent is administered to the subject orally.

Also disclosed are any preceding method, wherein the eosinophil protein-specific echogenic contrast agent is administered to the subject in an aerosol.

Also disclosed are any preceding method, wherein the subject is a mammal.

Also disclosed are any preceding method, wherein the mammal is a human.

Also disclosed are any preceding method, wherein the mucosal tissue of the esophagus comprises luminal surface of the esophagus.

Also disclosed are any preceding method, wherein the eosinophil protein-specific echogenic contrast agent is detected using ultrasound.

In another aspect, disclosed herein are methods of producing a medical image of an esophagus in a subject, comprising a) administering to a subject an eosinophil protein-specific echogenic contrast agent, wherein the eosinophil protein-specific echogenic contrast agent binds to a eosinophil protein in the mucosal tissue of the esophagus forming a contrast agent-eosinophil protein complex and b) detecting the presence of contrast agent-eosinophil protein complex in the mucosal tissue of the esophagus by ultrasound; and wherein detecting the echogenic contrast agent produces an image of the esophagus.

Also disclosed are any preceding method, wherein the ultrasound image is a two dimensional ultrasound image.

Also disclosed are any preceding method, wherein the ultrasound image is a three dimensional ultrasound image.

Also disclosed are any preceding method, wherein the eosinophil protein-specific echogenic contrast agent comprises an echogenic insulin aggregate linked to a targeting moiety specific for or that binds to an eosinophil protein.

Also disclosed are any preceding method, wherein the eosinophil protein is an eosinophil granule protein.

Also disclosed are any preceding method, wherein the eosinophilic peptide or protein comprises MBP-1, MBP-2, EDN, ECP, or EPO.

Also disclosed are any preceding method, wherein the eosinophil granule protein is MBP-1.

Also disclosed are any preceding method, wherein the eosinophil protein specific targeting moiety is linked to the echogenic insulin aggregate via a biotin-streptavidin interaction, wherein the targeting moiety is biotinylated and the insulin aggregate conjugated with streptavidin.

Also disclosed are any preceding method, wherein the targeting moiety is an antibody, polyglutamic acid, polyaspartic acid, or heparin.

Also disclosed are any preceding method, wherein the eosinophil protein-specific echogenic contrast agent is administered to the subject orally.

Also disclosed are any preceding method, wherein the eosinophil protein-specific echogenic contrast agent is administered to the subject in an aerosol.

In another aspect, disclosed herein are methods of monitoring the progression of eosinophilic esophagitis in a subject diagnosed with eosinophilic esophagitis, comprising: a) producing a first medical image of the esophagus of the subject according to any preceding method; b) producing a second medical image of the esophagus in the subject of step (a) according to any preceding method; and c) comparing the medical image of step (b) with the medical image of step (a); wherein an increase in the amount of bound contrast agent in the mucosal tissue of the esophagus in the medical image of step (b) relative to the medical image of step (a) indicates that the eosinophilic esophagitis has worsened; and wherein a decreases in the amount of bound contrast agent in the mucosal tissue of the esophagus in the medical image of step (b) relative to the medical image of step (a) indicates that the eosinophilic esophagitis has regressed.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight; temperature is in ° C. or is at ambient temperature; and pressure is at or near atmospheric.

### Example 1

### MBP-1 Specific Ultrasound Contrast Agent

The contrast agent was synthesized by preparing 2 mg/mL of insulin (Sigma, 12643) in HCl (pH 1.6) at 65° C. for 12 hours. The resulting particles were incubated with maleimide activated streptavidin (Pierce, 21102). Antibodies specific to MBP-1 (anti-MBP) was prepared as described previously. Anti-MBP-1 supernatant was purified using MabTrap Kit (Sigma, 54842). Purified anti-MBP was biotinylated using Pierce biotinylation protocol (TR0015.2) and reacted with the maleimide activated streptavidin insulin solution to form an insulin-maleimide-steptavidin-biotinylated-antiMBP-1 (hereafter referred to as insulin complex). Insulin complex comprises the MBP-1 specific ultrasound contrast agent (See FIG. 2).

### Example 2

### Monkey Esophagus

Macaca mulatta monkey esophagi purchased from California National Primate Research Center, incubated overnight in MBP-1 (0.27 mg/mL in 1×PBS), were filled with 2 mg/mL insulin complex solution. Monkey esophagus not treated with MBP-1 was used as control samples. For peristalsis experiments, a peristalsis pump at flow rate of 2 mL/min is used to pump the insulin/insulin complex through the monkey esophagus treated with/without MBP-1.

### Example 3

### Ultrasound Imaging

All images of the esophagus were obtained on a Siemens S 2000 platform using a high-resolution linear transducer with 8 to 15 MHz capability. Images were obtained at 14 MHz (linear & convex transducers 18L6, 576 elements, longitudinal and axial resolution <0.5 and <0.7 mm) The focal zone was set mid lumen of the esophagus and the field of view was the smallest possible. The platform preset for thyroid imaging was selected for consistency. Once the image quality was maximized for the control sample no further adjustments were made during the experiments.

### Example 4

### Insulin Aggregates

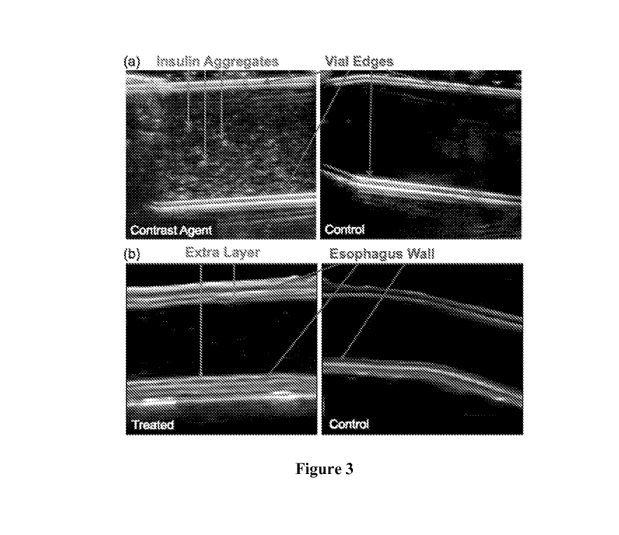

The prepared insulin samples were observed using ultrasound (see FIG. 3a). The insulin aggregates were clearly visible with high contrast compare to the control (water only). This indicates that insulin can be used as an ultrasound contrast agent.

### Example 5

### Insulin Complex with MBP-1 Treated Monkey Esophagus

Macaca Mulatta monkey esophagi, incubated overnight in MBP-1 were filled with insulin complex solution. After incubation for 5 minutes, the inner surface of the monkey was rinsed with water and ultrasound picture was taken. The results showed an extra contrast layer at the inner wall of the esophagus, demonstrating the specific binding of these agents to eosinophil granule proteins (FIG. 3b). No such layer is visualized in control samples (not treated with MBP-1). The extra line on the bottom of the treated sample in FIG. 3b is the reflection from the surface where the monkey was rested at the time of experiment.

### Example 6

### Peristalsis Experiment

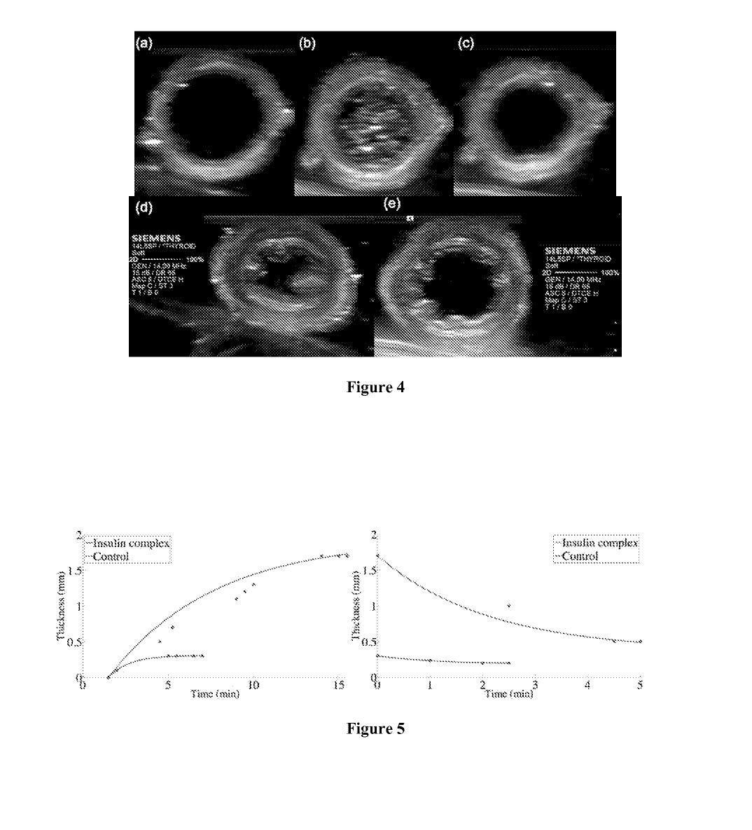

The binding of the insulin complex to MBP-1 likely depends on the esophageal peristalsis. In the peristalsis experiment, the insulin/insulin complex flowed through a peristaltic pump to the monkey esophagus coated with MBP-1. Real-time binding of the insulin particles to the esophageal wall was studied with ultrasound. FIG. 4 shows the transverse view of the monkey esophagus coated with MBP-1 with water/insulin/insulin complex pumped through. The esophagus walls were visualized in ultrasound image with flowing water through the esophagus (FIG. 4a). When pumping the insulin aggregates only, the bright insulin particles were visualized in the transverse view of the esophagus (FIG. 4b). As shown in figure, the inner wall intensity slightly increased after 5 minutes flow of insulin aggregates. However, after the system was washed with water (FIG. 4c), the contrast intensity decreased, but still higher maringally than the control (FIG. 4a). The results showed a slight increase in the contrast intensity at the inner surface of the esophagus, indicating non-specific binding of the insulin aggregates to the tissue. FIG. 4d, shows the flow of insulin complex through the monkey esophagus treated with MBP-1 for 5 minutes. The results clearly showed that there was a distinct extra layer at the inner surface of the esophagus. The extra layer was still distinguishable after washing step with water (FIG. 4e), indicating that the insulin complex binds significantly to MBP-1 under continuous flow through the tissue.

### Example 7

### Binding Coefficients

To understand the rate of binding of insulin/insulin complex to the monkey esophagus tissue treated with MBP-1, the thickness of the insulin complex layer was measured over time. FIG. 5, shows the thickness of insulin complex measured over the time compared to control (insulin only). The insulin complex thickness gradually increased to over 1.5 mm, whereas the control sample thickness was not very significant (FIG. 5). After flowing water through the esophagus in both cases, the insulin complex thickness was double that of the control sample, even after 5 minutes.

The surface reaction of insulin complex can be expressed as

$$\frac{d {N}_{b}}{d t} = {k}_{on} {N}_{f} {N}_{site} - {k}_{off} {N}_{b} \frac{d {N}_{b}}{d t} = {k}_{on} {N}_{f} {N}_{site} - {k}_{off} {N}_{b} , \qquad ( 1 )$$

where N_{b} is the number of particles bounded to the MBP-1 surface at any given time, N_{f} is the number of free particles floating in a slurry motion near the wall, and Nₛᵢₜₑ is the number of sites available for binding, which is the ratio of esophagus length to the diameter of particles, L and dₚ respectively. N_{b} can be expressed as the area for the experimentally measured thickness (h) divided by individual surface of each particle.

$${N}_{b} = \frac{4 hL}{π {d}_{p}^{2}} \qquad ( 2 )$$

Simplifying equation 1 to:

$$\frac{d h}{d t} = {k}_{on} \frac{π {d}_{p} {N}_{f}}{4} - {k}_{off} h \qquad ( 3 )$$

In this example, the experimental values for N_{f} and dₚ are 8 and 0.8 mm, respectively. The analytical solution to the above equation is

$$h = 0.0265 \frac{{k}_{on}}{{k}_{off}} - {c}_{1} exp ( - {k}_{off} t ) , \qquad ( 4 )$$

where c₁ is the integral constant, which can be calculated experimentally from the boundary conditions. To calculate the binding coefficients, Equation 4 was solved to fit the experimental data points in FIG. 4. The maximum thickness measured, kₒₙ, and k_{off} for insulin and insulin complex are summarized in Table 1. In both cases, kₒₙ values are very close, however, the k_{off} for insulin only is 8 and 2 times higher in binding and washing step, respectively.

TABLE 1
Summarized value of binding coefficients and maximum
thickness for binding and washing step.

| | Binding Step | | Washing Step | |
|---|---|---|---|---|
| | Insulin | Insulin | Insulin | Insulin |
| | Complex | Only | Complex | Only |
| Maximum Thickness (mm) | 1.7 | 0.3 | 0.6 | 0.2 |
| kₒₙ (min⁻¹) | 0.06 | 0.05 | 0.04 | 0.03 |
| k_{off} (min⁻¹) | 0.16 | 0.83 | 0.48 | 0.84 |

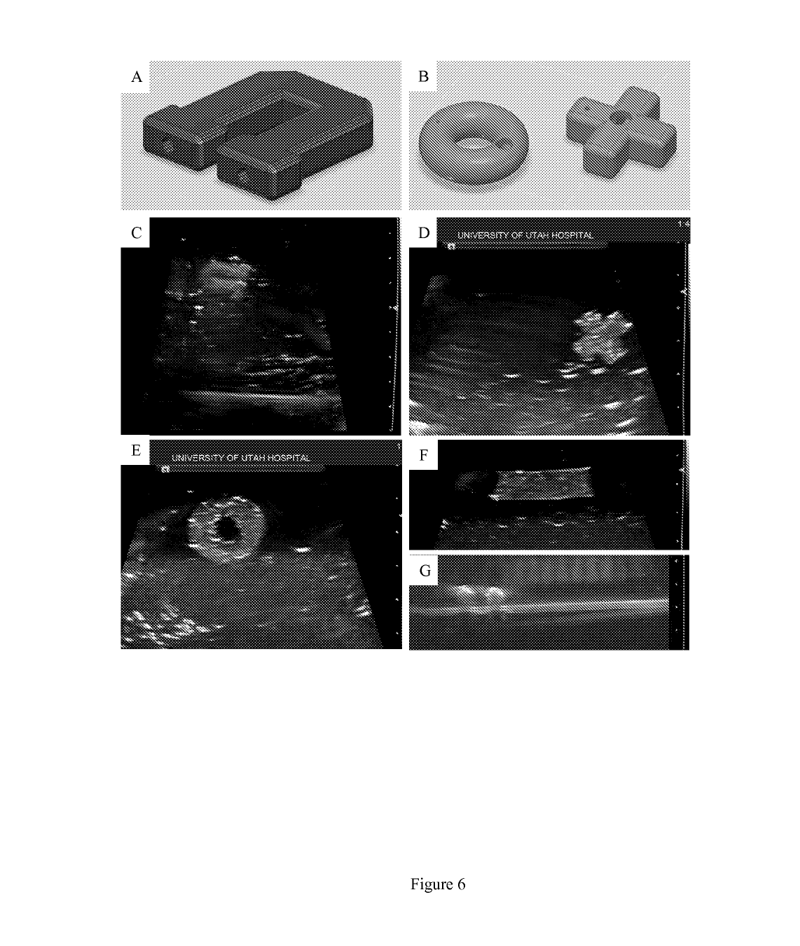

Images of molds and solid polymer shapes were first designed graphically as shown in FIGS. 6A and 6B. Crosses, toroids, and the letter U were selected for demonstration purposes. These structures were then printed in poly(lactic acid) (PLA) using a 3D printer (MakerBot Replicator 2.0). At least one version of each structure contained ports to allow for filling. The ports were filled with insulin particle solution, perfluorooctane, or air (i.e., left empty). The insulin solution was prepared by incubating insulin at 1-3 mg/mL at 65-85° C. for 2-24 hours. The filled particles were then sealed and imaged using ultrasound. Imaging was performed using a Siemens S 2000 platform using a high-resolution linear transducer with 8 to 15 MHz capability. Images were obtained at 14 MHz. FIGS. 6C and 6D depict an insulin filled letter U and a solid PLA letter U as a control both in bubble rich environments. The shapes are clearly visible and contrast clearly with the smaller bubbles in its vicinity. FIG. 6E depicts a donut shaped torus in cross section imaged using ultrasound. This shape was filled with insulin confirming that insulin filled configurations appear brightly. FIG. 6F compares air and perfluorooctane 2D crosses. The perfluorooctane filled structure is brighter as anticipated, while the air filled structure remains less distinct but clearly visible. Taken together, these images demonstrate that shape defined contrast agents can be distinctly imaged using ultrasound. These are important results because they indicate that multiple shape defined contrast agents can be imaged from the same solution. This enables multiplexed analysis of mucosal and lumenal diseases.

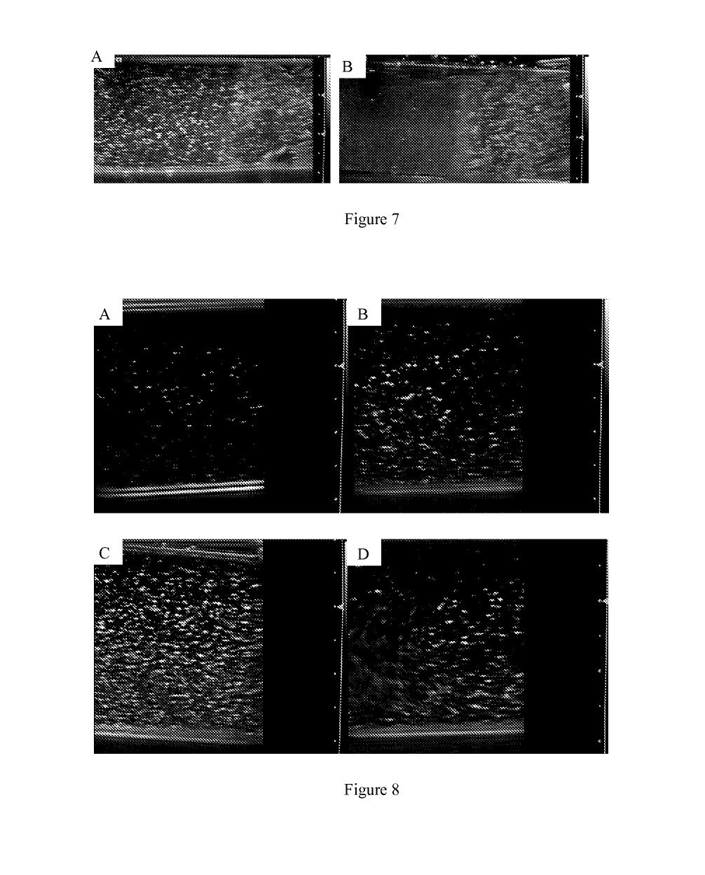

An important feature of these solid particles is that they are readily apparent at interfaces. For example, FIG. 7A compares the contrast provided by the insulin particles at water-tissue simulant interfaces. Here the tissue simulant has a pudding-like consistency. Insulin particles are readily apparent on both sides of the interfaces, though they blur slightly on the pudding (right) side of the white dashed line representing the interface. FIG. 7B further shows that the insulin particles remain in the tissue simulant when the water does not contain particles. Taken together with the previous figure, these figures indicate that the particle disclosed herein provide distinct contrast from or/and at interfaces.

FIG. 8 depicts size separated insulin particles. These particles were separated using a series of sieve trays of approximately 25 micron intervals. For example, FIG. 8A represents particles approximately 50-75 microns size, FIG. 8B represents particles approximately 75-100 microns in size, and FIG. 8C represents particles approximately greater than 100 microns in size. Taken together, these images show that insulin particles of a variety of sizes provide distinctive contrast in this modality. However, the larger particles appear brighter than the smaller particles that provide contrast by scattering only. FIG. 8D shows that flow affects the particles and that the particles follow lines of flow.

With the advent of 3D printing, tagging one surface of a 3D structure for one biomarker and other surfaces of the same structure for a different biomarker can be done. For example, a pyramid (triangular faces but square bottom) can be printed out of PLA on a PVA mold. The subject exposed triangular or square faces can then be added to one targeting moiety such that this moiety is available on the said faces for binding to a disease marker. PVA layers ˜½ mm thick tend to dissolve slowly over 30-50 minutes. During this time, the moiety can bind and reactants be removed. Protecting groups can be used. After the PVA completely dissolves, a second reactive moiety can then be introduced such that the surfaces previously covered by the PVA become decorated by this second moeity. In this way Janus particles can be formed. When the composition is a 3D rectangle, for example, then the longest sides can bind to one disease target while the smallest sides can indicate a different disease target along the surface of the lumen. In this manner, disease detection can be multiplexed by particle orientation. For example, esophageal cancer and EoE or two small bowel diseases can be simultaneously screened with one type of particle.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

1. D. Savery, G. Cloutier, High-frequency ultrasound backscattering by blood: analytical and semianalytical models of the erythrocyte cross section. J Acoust Soc Am 2007, 121. 3963-71.
2. (a) A. Bertram, K. Ley, Protein kinase C isoforms in neutrophil adhesion and activation. Arch Immunol Ther Exp (Warsz) 2011, 59. 79-87; (b) N. M. Green, A. Marshak-Rothstein, Tolllike receptor driven B cell activation in the induction of systemic autoimmunity. Semin Immunol 2011, 23. 106-12.
3. H. Kita, Eosinophils: multifaceted biological properties and roles in health and disease. Immunol Rev 2011, 242. 161-77.
4. V. V. Mody, R. Siwale, Application of nanoparticles in diagnostic imaging via ultrasonography. Internet Journal of Medical Update 2011, 6. 8-15.
5. R. Shamir, S. Kolacek, S. Koletzko, I. Tavori, D. Bader, I. Litmanovitz, O. Flidel-Rimon, K. A. Marks, I. Sukhotnik, N. Shehadeh, Oral insulin supplementation in paediatric short bowel disease: a pilot observational study. J Pediatr Gastroenterol Nutr 2009, 49. 108-11.
6. A. R. Abdul Latif, R. Kono, H. Tachibana, K. Akasaka, Kinetic analysis of amyloid protofibril dissociation and volumetric properties of the transition state. Biophys J 2007, 92. 323-9.
7. G. Furuta, A. Kagawalla, P. Alumkal, The Esophageal String Test (EST): A novel minimally invasive method for measuring esophageal inflammation in eosinophilic esophagitis. 7th Biennial Symposium of the International Eosinophil Society Jun. 23, 2011. Poster E-12.
8. H. Saffari, K. A. Peterson, J. Fang, C. Teman, G. J. Gleich, L. F. Pease, Patchy Eosinophil Distributions in Eosinophilic Esophagitis Esophagectomy Specimen: Implications for Endoscopic Biopsy. American Journal of Gastroenterology 2011. Submitted.
9. G. J. Gleich, Mechanisms of eosinophil-associated inflammation. J Allergy Clin Immunol 2000, 105. 651-63.
10. L. F. Pease, M. Sorci, S. Guha, D.-H. Tsai, M. R. Zachariah, M. J. Tarlov, G. Belfort, Probing the Nucleus Model for Oligomer Formation During Insulin Amyloid Fibrilogenesis. Biophysical Journal 2010, 99. 3979-3985.

What is claimed is:

1. An echogenic contrast agent comprising an targeting moiety linked to an echogenic insulin aggregate.

2. The echogenic contrast agent of claim 1, wherein the targeting moiety is specific for or binds to an eosinophilic peptide or protein present in the mucosal tissue surrounding an eosinophil.

3. The echogenic contrast agent of claim 2, wherein the eosinophilic peptide or protein comprises major basic protein 1 (MBP-1), major basic protein 2 (MBP-2), eosinophil derived neurotoxin (EDN), eosinophil cationic protein (ECP), or eosinophil peroxidase (EPO).

4. The echogenic contrast agent of claim 1, wherein the targeting moiety is an antibody or an anionic heparin.

5. The echogenic contrast agent of claim 1, wherein the targeting moiety is biotinylated.

6. The echogenic contrast agent of claim 1, wherein the echogenic insulin aggregate is conjugated with streptavidin.

7. The echogenic contrast agent of claim 1, wherein the insulin aggregate has a cylindrical, circular, star, cross, rod, rectangular, smiley face, frowny face, or ellipsoidal shape.

8. A method of diagnosing eosinophilic esophagitis in a subject, comprising administering to a subject an eosinophil protein-specific echogenic contrast agent and detecting the presence of bound contrast agent in the mucosal tissue of the esophagus, wherein the eosinophil protein-specific echogenic contrast agent binds to a eosinophil protein in the mucosal tissue of the esophagus; and wherein the presence of bound contrast agent in the mucosal tissue of the esophagus indicates that the subject has eosinophilic esophagitis.

9. The method of claim 8, wherein the eosinophil protein-specific echogenic contrast agent comprises an echogenic insulin aggregate linked to a targeting moiety specific for or that binds to an eosinophil protein.

10. The method of claim 9, wherein the eosinophil protein is an eosinophil granule protein.

11. The method of claim 10, wherein the eosinophil granule protein comprises MBP-1, MBP-2, EDN, ECP, or EPO.

12. The method of claim 11, wherein the eosinophil granule protein is MBP-1.

13. The method of claim 9, wherein the eosinophil protein specific targeting moiety is linked to the echogenic insulin aggregate via a biotin-streptavidin interaction, wherein the targeting moiety is biotinylated and the insulin aggregate conjugated with streptavidin.

14. The method of claim 9, wherein the targeting moiety is an antibody or heparin.

15. The method of claim 8, wherein the eosinophil protein-specific echogenic contrast agent is administered to the subject orally.

16. The method of claim 8, wherein the eosinophil protein-specific echogenic contrast agent is administered to the subject in an aerosol.

17. The method of claim 8, wherein the subject is a mammal.

18. The method of claim 8, wherein the mammal is a human.

19. The method of claim 8, wherein the mucosal tissue of the esophagus comprises luminal surface of the esophagus.

20. The method of claim 8, wherein the eosinophil protein-specific echogenic contrast agent is detected using ultrasound.

21. A method of producing a medical image of an esophagus in a subject, comprising
a) administering to a subject an eosinophil protein-specific echogenic contrast agent, wherein the eosinophil protein-specific echogenic contrast agent binds to a eosinophil protein in the mucosal tissue of the esophagus forming a contrast agent-eosinophil protein complex and
b) detecting the presence of contrast agent-eosinophil protein complex in the mucosal tissue of the esophagus by ultrasound; and wherein detecting the echogenic contrast agent produces an image of the esophagus.

22. The method of claim 21, wherein the ultrasound image is a two dimensional ultrasound image.

23. The method of claim 21, wherein the ultrasound image is a three dimensional ultrasound image.

24. The method of claim 21, wherein the eosinophil protein-specific echogenic contrast agent comprises an echogenic insulin aggregate linked to a targeting moiety specific for or that binds to an eosinophil protein.

25. The method of claim 24, wherein the eosinophil protein is an eosinophil granule protein.

26. The method of claim 25, wherein the eosinophilic peptide or protein comprises MBP-1, MBP-2, EDN, ECP, or EPO.

27. The method of claim 26, wherein the eosinophil granule protein is MBP-1.

28. The method of claim 24, wherein the eosinophil protein specific targeting moiety is linked to the echogenic insulin aggregate via a biotin-streptavidin interaction, wherein the targeting moiety is biotinylated and the insulin aggregate conjugated with streptavidin.

29. The method of claim 24, wherein the targeting moiety is an antibody or heparin.

30. The method of claim 21, wherein the eosinophil protein-specific echogenic contrast agent is administered to the subject orally.

31. The method of claim 21, wherein the eosinophil protein-specific echogenic contrast agent is administered to the subject in an aerosol.

32. A method of monitoring the progression of eosinophilic esophagitis in a subject diagnosed with eosinophilic esophagitis, comprising:
a) producing a first medical image of the esophagus of the subject according to the method of claim 8;
b) producing a second medical image of the esophagus in the subject of step (a) according to the method of claim 8; and
c) comparing the medical image of step (b) with the medical image of step (a); wherein an increase in the amount of bound contrast agent in the mucosal tissue of the esophagus in the medical image of step (b) relative to the medical image of step (a) indicates that the eosinophilic esophagitis has worsened; and wherein a decreases in the amount of bound contrast agent in the mucosal tissue of the esophagus in the medical image of step (b) relative to the medical image of step (a) indicates that the eosinophilic esophagitis has regressed.
